(19) United States
(12) Patent Application Publication
(10) Pub. No.: US 2021/0267752 A1
(43) Pub. Date: Sep. 2, 2021

(54) OPHTHALMOLOGICAL IMPLANT COMPRISING AN ACTIVE INGREDIENT RELEASE SYSTEM AND METHOD FOR PRODUCING AN OPHTHALMOLOGICAL IMPLANT OF THIS TYPE

(71) Applicant: Carl Zeiss Meditec AG, Jena (DE)

(72) Inventor: Michael Thaller, Berlin (DE)

(21) Appl. No.: 17/326,016

(22) Filed: May 20, 2021

Related U.S. Application Data
(63) Continuation of application No. PCT/EP2019/080503, filed on Nov. 7, 2019.

(30) Foreign Application Priority Data
Nov. 22, 2018 (DE) 10 2018 129 478.6

(51) Int. Cl. A61F 2/16; A61L 27/52; A61L 27/54; A61L 27/26; A61L 27/34

(52) U.S. Cl. CPC A61F 2/16; A61L 27/52; A61L 27/54; A61L 27/26; A61L 27/34; A61L 2420/02; A61F 2250/0067; A61L 2400/12; A61L 2430/16; A61L 2300/41; A61F 2240/001

(57) ABSTRACT

Provided are ophthalmological implants comprising an active ingredient release system which, when the ophthalmological implant is implanted, delivers at least one pharmacological active ingredient, the active ingredient release system comprising at least one hydrogel as a matrix, the hydrogel forming a layer on an optical portion of the implant, binding covalently to the optical portion and being charged with the at least one active ingredient. Also provided are methods for producing the ophthalmological implant comprising the active ingredient release system.

### CROSS REFERENCE TO RELATED APPLICATIONS

This application is a continuation application of international patent application PCT/EP2019/080503, filed Nov. 7, 2019, designating the United States and claiming priority from German application 10 2018 129 478.6, filed Nov. 22, 2018, and the entire content of both applications is incorporated herein by reference.

### BACKGROUND

Fibrosis refers to a pathological increase in connective tissue in human and animal tissues, the main component of which is collagen fibers. This hardens the tissue of the organ affected. The result is scar-like lesions which, at an advanced stage, lead to restriction of the particular function of the organ. In the case of use of ophthalmological implants, for example intraocular lenses (IOLs), interactions between the implant and adjacent biological tissue mean that various complications can occur, of which such fibroses in particular are problematic. For example, so-called posterior capsule opacification (PCO, cataracta secundaria) occurs in some cases after cataract operations. PCO is a post-operative clouding of the lens capsule after surgical extraction of a natural lens. The remaining lens epithelial cells (E cells) in the equatorial region of the capsular bag are mitotically active and can transform into fibroblasts. These then trigger a kind of wound healing, involving formation of collagen-containing connective tissue. Since some fibroblast subtypes not only migrate onto the inner side of the capsular bag, but can also contract, wrinkles form in the capsular bag. The clouding of the capsule is therefore the result of a wound healing process and associated scarring. Since the lens clouding caused thereby has causes other than the original cataract disease, this is referred to as an “aftercataract” or a “secondary cataract”. For those affected, a clinically significant aftercataract can lead to a reduction in visual acuity, in color perception and in contrast vision and to increased glare.

Secondary cataract is a common complication after extracapsular cataract extraction (ECCE) and the subsequent implantation of an intraocular lens (IOL) in the capsular bag. Without the implantation of an IOL in the empty capsular bag, the risk of an aftercataract is even more considerably increased, since unhindered cell migration to the posterior surface of the capsular bag is possible in this case.

The incidence of a secondary cataract increases over time after a surgical procedure. A meta-analysis of the cases of cataracta secundaria for all existing types of IOLs showed an approximate average increase of 12% one year after surgery and an approximate average increase of 30% five years after surgery. The age of the affected patient also appears to be a crucial factor here, and so it has to be expected that almost all treated children and adolescents might suffer from a post-operative secondary cataract after a certain period of time.

A further common post-operative complication of cataract operation is eye inflammation. This is caused by the wound in the cornea that results from the corneal incision required for the replacement of the natural lens by a synthetic implant. This physical trauma can trigger an inflammation reaction in that it releases mediators of inflammation such as prostaglandins and leukotrienes that subsequently trigger an immune response within the eye. This can quickly lead to pain and complaints in the patient. In the case of improper treatment, lasting inflammation can lead to serious side effects such as posterior synechiae, uveitis and secondary glaucoma. It is therefore important to manage post-operative operation as well as possible.

Post-operative inflammation management is typically implemented with medicaments. There are currently two recognized classes of active ingredient: one based on corticosteroids and one based on “non-steroidal anti-inflammatory drugs” (NSAIDS). The steroidal medicaments are generally considered to be more effective, but have side effects, for example a rise in intraocular pressure, whereas the NSAIDS cause fewer complications but are generally less effective, and studies on this topic are not yet complete.

A certain problem is also the necessity of patient compliance. Inflammation-inhibiting medicaments generally have to be prescribed for 4-6 weeks, and during this time have to be administered by the patient themself via eyedrops. However, it is common for patients to be unable to keep to their medicament regime. Reasons for this may include, for example, that they do not see the necessity of the treatment, do not want to or cannot pay the costs of the medicament, are physically incapable of applying the medicaments correctly themselves, or forget to administer them. This lack of compliance with the medicament plan is often observed in more than half of patients. Even in the case of regular studies, this lack of compliance with the medicament plan is, however, often not apparent to a doctor. There are also some regions in which patients, for example owing to a long travel time, cannot regularly come into a practice for examination. These shortcomings can lead to problems in post-operative treatment and to patient discomfort. It is therefore very desirable to dispense with active involvement of the patient as far as possible in the implementation of a post-operative medicament plan, and to find a way of ensuring correct administration of medicaments. This should preferably be done in such a way that such a solution is or can be integrated into the normal course of an eye operation.

WO 2008/113043 A1 discloses an ophthalmological implant configured as an intraocular lens having one optical component and two tactile components. An active ingredient release system is disposed on at least one of the tactile components, in order to be able to release an active pharmaceutical ingredient in the implanted state of the implant.

A disadvantage of the known ophthalmological implant is considered to be the fact that the amount of active ingredient that can be released is comparatively small since only thin tactile components are used, unless the release system itself is relatively bulky. However, this would increase the necessary incision size in the implantation and make the lens less safe for surgical use. Moreover, this would make it more likely that the functionality of the tactile components and hence of the optical component as well will be impaired.

### SUMMARY

Described herein are ophthalmological implants that provide improved and longer-lasting active ingredient release. Production of such ophthalmological implants is also described herein.

Provided are ophthalmological implants that comprise an active ingredient release system configured to release at least one pharmacologically active ingredient in the implanted state of the ophthalmological implant. Improved and longer-lasting active ingredient release is provided by these implants since the active ingredient release system of the implants comprises at least one hydrogel as a matrix, wherein the hydrogel forms a layer on an optical component of the implant, is covalently bonded to the optical component, and is laden with the at least one active ingredient. In other words, the active ingredient release system of the implants described herein is disposed as a coating on the optical component of the implant and is immobilized on the optical component by covalent binding of the hydrogel. The hydrogel generally functions as a matrix for the active ingredient to be released, with which the hydrogel is laden. By contrast with an arrangement on a tactile component, this enables use of a significantly greater area of the implant for the arrangement of the active ingredient release system with a correspondingly greater amount of active ingredient without having to accept restrictions with regard to the space required, implantability and implant functionality. Since hydrogels in the hydrated state by definition contain a comparatively large amount of water, or can frequently absorb several times their own weight of water on contact with water, their refractive index in the implanted and hydrated state is comparable to that in the environment of the eye. Therefore, hydrogels are virtually invisible to the human and animal eye. This enables application of the hydrogel in the form of a layer as a matrix of the active ingredient release system, that is, in two-dimensional form and not in the form of isolated particles or the like, to the optical surface of the implant, since it does not impair the optical properties of the optical component. Furthermore, it is possible in this way to modify implants with the active ingredient release system independently of any tactile component, that is, for example, implants having only one tactile component or implants having no tactile components. If the implant has one or more tactile components, it may be the case that the tactile component(s) is/are configured without an active ingredient release system. Alternatively, the active ingredient release system is in certain embodiments, in addition to the optical component, also configured in one or more tactile components. The active ingredient release system is in some embodiments, in principle, disposed on one or more surface regions of the optical component or over the entire surface of the optical component. For example, the hydrogel covers 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the surface area of the optical component. In addition, the active ingredient release system in some embodiments forms a single layer or multiple layers, that is, it is formed from two or more layers in some embodiments. In the case of multiple layers, these layers each have the same composition or different compositions, have the same or different thickness, and contain identical or different active ingredients or active ingredient combinations. The two or more layers are likewise in some embodiments covalently coupled to one another in order to assure particularly reliable binding of the overall layer system to the optical component. The total thickness of the hydrogel layer(s) is, in particular applications, up to 200 μm, that is, for example, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In principle, however, greater layer thicknesses are envisaged in certain embodiments, for example 200 μm, 250 μm, 300 μm, 350 μm, 400 μm, 450 μm, 500 μm, or more, where corresponding intermediate values should be considered to be included in the disclosure. An “active ingredient” as used herein is generally a pharmacologically active substance, optionally in the form of its salt, of a conjugate, et cetera, and/or a precursor substance (“prodrug”) that only becomes active after metabolization. In general, “a/an” in the context of this disclosure is to be read as an indefinite article, that is, always as “at least one” if there is no express indication of the contrary. Conversely, “a/an” can also be understood to mean “just one”.

In an advantageous embodiment, the implant is configured as an intraocular lens, such as an accommodating intraocular lens, ring, such as a capsular tension ring, or a tube. As a result, the advantages realizable according to the embodiments described herein are realized in the context of different eye operations and implant types. This is particularly true of an implant in the form of an accommodating intraocular lens (IOL) since, in the case of such an IOL, inflammation reactions, fibroses, and the like, could impair the accommodation ability of the IOL or completely prevent it from doing so. Furthermore, the ophthalmological implant is in some embodiments configured as a tube, such as a dialysis tube. For a surgical insertion, intraocular lenses are typically folded and injected into the eye with an injector. In order to limit any possible damage to the cornea, it is desirable to reduce the incision size as far as possible and to keep the injection tips of the injector as small as possible. The effect of this is that a certain expenditure of force is required to force the folded IOL through the injector. These forces are generally reduced by glide coating of the inner wall of the injector. Since the implant of the invention has been coated with a hydrogel, the medicament-releasing hydrogel of the active ingredient release system advantageously provides additional lubrication and hence either improves the existing injection process or can even completely replace, or render superfluous, the existing glide coating on the injector wall.

In a further advantageous configuration described herein, the hydrogel is degradable, or biodegradable. This enables particularly readily controllable release of the at least one active ingredient stored in the hydrogel, which allows unwanted released peaks to be particularly reliably prevented and the active ingredient to be released continuously over a period of several weeks. The hydrogel is degraded, for example, by virtue of the hydrogel having hydrolyzable polymer bonds and/or hydrolyzable chemical bonds to the active ingredient molecules. Alternatively or additionally, the hydrogel has in some embodiments, enzymatically degradable polymer bonds and/or enzymatically degradable bonds to the active ingredient molecules. For example, it is possible to provide peptide bonds that are cleaved by peptidases after implantation. The release of the active ingredient can be configured here such that the active ingredient can be released exclusively through degradation of the hydrogel, for example in that the active ingredient is bound to the hydrogel, and/or in that the active ingredient is a sterically “trapped” in the lattice of the hydrogel matrix. In some embodiments, the active ingredient, in addition to release by degradation of the hydrogel, is also released by diffusion out of the hydrogel, which generally enables a higher release rate.

In a further advantageous configuration, the hydrogel is selected from one or more of the following: poly(N-isopropylacrylamide), polyvinylalcohol, polyethyleneglycol, polylactic acid, polyethyleneimine, cellulose, cellulose ethers having methyl and/or ethyl and/or propyl groups, especially hydroxypropyl methylcellulose, hydroxyethyl methylcellulose and/or methylcellulose, glycosaminoglycans, especially hyaluronic acid, chondroitin sulfate, dermatan sulfate, heparin, heparan sulfate, keratan sulfate, alginic acid, polymannuronic acid, polyguluronic acid, polyglucuronic acid, amylose, amylopectin, callose, chitosan, polygalactomannan, dextran, xanthan and/or a mixture and/or a physiologically acceptable salt thereof. It is thus possible to vary the properties of the hydrogel, for example its viscoelasticity and degradability, to the respective intended use and purpose of the implant. Hydrophilic long-chain polymers can be converted to hydrogels, for example, by simple crosslinking reactions. Owing to the large amount of hydrophilic groups, the gel can subsequently absorb a correspondingly large amount of water, often several times its own weight. The same interactions are utilized in some embodiments to physically trap active ingredient molecules within the hydrogel network. For slow and long-lasting release, hydrophilic and comparatively large or sterically demanding active ingredient molecules are contemplated, since they do not diffuse as quickly out of the hydrogel and reduce the likelihood of a burst release of a majority of the amount of active ingredient present shortly after implantation. Small hydrophobic active ingredients are also in some embodiments successfully trapped in such networks, for example by copolymerization with other monomers or by manipulation of the polymer surface. Alternatively, such active ingredient molecules are covalently bonded to the polymer via a degradable bond. In that case, it is the degradation rate that controls the release of the active ingredients rather than the diffusion of these molecules. Polysaccharides, which are also referred to as glycans, are in general a subclass of carbohydrates and are polysugars composed of monosaccharide units (for example, glucose, fructose, galactose, et cetera) which form a chain. Every monosaccharide, which is also referred to as simple sugar, consists of a chain of carbon atoms. Depending on the number of carbon atoms, reference is made to trioses (3), tetroses (4), pentoses (5), hexoses (6), heptoses (7), et cetera. Depending on the nature of their saccharide units, the polysaccharides can be divided into homoglycans, which have only one type of simple sugar, and heteroglycans, which have two or more different types of simple sugars. Furthermore, polysaccharides can be unsubstituted or substituted and can bear one or more side groups, for example hydroxyl, carboxy, amino or sulfate groups.

In a further advantageous configuration, the at least one active ingredient is covalently bonded to the hydrogel via a degradable or biodegradable type of bond. This likewise enables particularly readily controllable release of the at least one active ingredient bound to the hydrogel, which allows unwanted released peaks to be particularly reliably prevented and the active ingredient to be released continuously over a period of several weeks. The controlled release of the active ingredient can also be implemented in conjunction with a non-degradable or sparingly degradable hydrogel. The degradation of the covalent bond and the release of the active ingredient in this case too is effected, for example, in that the active ingredient has hydrolyzable chemical bonds to the hydrogel network. Alternatively or additionally, the active ingredient has enzymatically degradable bonds to the hydrogel network. For example, it is possible to provide peptide bonds that are cleaved by peptidases after implantation. Alternatively or additionally, the active ingredient is covalently bonded to a monomer and/or oligomer. This increases the space requirement of the active ingredient, and so even small active ingredient molecules, by virtue of steric hindrance, are able to diffuse out of the hydrogel network only at low rates, if at all. The monomer or oligomer in some instances does not impair the pharmacological action of the active ingredient. In some embodiments the bond of the active ingredient of the monomer/oligomer is degradable, such that the active ingredient is in its physiologically active form before and/or after diffusion out of the hydrogel network. In other embodiments the active ingredient is distributed in the hydrogel. This is achieved in that the active ingredient is present in the reaction mixture during the polymerization of the hydrogel reactants or precursors and is “trapped” in the network formed, and/or in that the hydrogel is introduced into a preferably saturated solution of the active ingredient, such that the active ingredient diffuses into the hydrogel. Alternatively or additionally, the active ingredient is present in polymer nanoparticles distributed within the hydrogel that are biodegradable in particular. The polymer nanoparticles in turn themselves, in certain embodiments, are comprised of a hydrogel and/or are degradable or biodegradable. In this way, it is possible to particularly precisely adjust the release rate of the active ingredient. In addition, the active ingredient is in such instances protected from unwanted reactions in the formation of the hydrogel network.

In a further advantageous configuration, at least one active pharmacological ingredient is covalently bonded to at least one surface region of the hydrogel. This allows the desired pharmacological action to be achieved directly at the implant surface. For example, the active ingredient is in some embodiments an antibody that captures and binds unwanted target structures, for example inflammation mediators, fibrogens, and the like, directly at the implant surface. This particularly reliably prevents impairments of the optical properties of the implant.

In a further advantageous configuration, the at least one active ingredient is selected from steroidal and non-steroidal inflammation inhibitors, especially COX-2 inhibitors, prostaglandins and/or prostamides, antibiotics, and beta-blockers. This allows the pharmacological action of the implant to be optimized to different expected endogenous reactions and potential complications, for example to inflammation reactions and/or PCOs.

In a further advantageous configuration, the implant is stored in a saturated solution of the at least one active ingredient. Especially in cases where the active ingredient is not firmly bonded to the hydrogel or is “trapped” in the hydrogel in some other way, but is to be released by diffusion, for example, this ensures that the concentration of the active ingredient in the active ingredient release system remains constant during the storage of the implant.

Also provided are processes for producing an ophthalmological implant having an active ingredient release system configured to release at least one pharmacologically active ingredient in the implanted state of the ophthalmological implant. An improved and longer-lasting release of active ingredient is provided in the described implants in that an optical component of the implant is coated with at least one hydrogel as matrix of the active ingredient release system and the at least one hydrogel is covalently bonded to the optical component, where the hydrogel is laden with the at least one active ingredient. In other words, the active ingredient release system is disposed as a coating on the optical component of the implant and is immobilized on the optical component by covalent binding of the hydrogel. The hydrogel generally functions as a matrix for the active ingredient to be released, with which the hydrogel is laden. By contrast with an arrangement on a tactile component, this allows use of a significantly greater area of the implant for the arrangement of the active ingredient release system with a correspondingly greater amount of active ingredient without having to accept restrictions with regard to the space required, implantability, and implant functionality. Since hydrogels in the hydrated state by definition contain a comparatively large amount of water, or can frequently absorb several times their own weight of water on contact with water, their refractive index in the implanted and hydrated state is comparable to that in the environment of the eye. Therefore, hydrogels are virtually invisible to the human and animal eye. The loading of the hydrogel with the active ingredient or active ingredient combination is generally effected before the coating of the optical component with the hydrogel, during the coating of the optical component with the hydrogel, and/or after the covalent binding of the hydrogel to the optical component. In some embodiments the hydrogel is first applied and covalently bonded to the optical component in the partly or fully dehydrated state and is then hydrated by contacting with water. The covalent attachment of the hydrogel is in some instances performed by any suitable chemical method, for example by what is called click chemistry.

In an advantageous configuration, the covalent binding of the at least one hydrogel to the optical component of the implant comprises the steps of generating surface hydroxyl groups on the optical component of the implant, graft polymerizing at least one reactive silane compound bearing at least one further functional group as well as a silane group onto the surface hydroxyl groups of the implant, and covalently binding the at least one hydrogel onto the further functional group of the grafted silane compound. The generating of surface hydroxyl groups on the optical component of the implant is general required only when the optical component itself has too small a number of free hydroxyl groups, if any. Hydroxyl groups are in some embodiments generated by plasma activation, although it is generally also possible to use other suitable techniques. Suitable silane compounds are for instance trialkoxysilanes of the general formula (I)

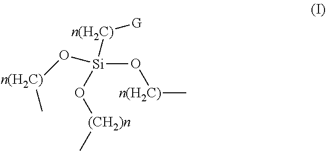

in which G denotes the functional group and the parameters n are selected independently of one another, that is, are 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more. G is, for example, an amino group or an ethenyl group, although other functional groups, for example carboxyl groups, epoxy groups, phosphate groups, anhydrides, hydroxyl groups, thiol groups, and the like, and corresponding combinations, that permit the covalent coupling of the hydrogel to the optical component are also possible. For example, the silane compound is in an embodiment 3-(triethoxysilyl)propan-1-amine or triethoxy(hex-5-enyl)silane. In the case of silane bearing amino groups and hydrogel bearing amino groups, covalent coupling is in some instances accomplished using crosslinkers bearing two or more carboxyl groups and optionally additionally containing one or more (bio)degradable bonds. Through use of a coupling reaction mediated by EDC (1-ethyl-3-(3-dimethylaminopropyl)carbodiimide hydrochloride)/NHS (1-hydroxy-2,5-pyrrolidinedione) that is known per se, it is then possible to couple the hydrogel covalently to the optical component via peptide bonds. The functional groups are of course in some embodiments also exchanged, meaning that the hydrogel bears carboxyl groups and the crosslinker bears amino groups. In some embodiments the silane bears a functional alkenyl group that is coupled via a crosslinker bearing at least two alkenyl groups (diene, triene, et cetera) to the hydrogel bearing thiol groups via a Michael-type thiol reaction. Depending on the functional groups present in each case, however, there are of course generally also other suitable crosslinking reactions that are conceivable.

The implant descriptions provided herein are considered to include and disclose embodiments that are not shown and elucidated explicitly in the figures, but result from and can be created from separate combinations of features from the details elucidated. The present description shall also be considered to extend to embodiments and combinations of features that thus do not have all the described features. Furthermore, the present description shall be considered to extend to embodiments and combinations of features, especially via the embodiments set out above, that go beyond or depart from the combinations of features set out herein.

### BRIEF DESCRIPTION OF THE DRAWINGS

The invention will now be described with reference to the drawings, wherein:

FIG. 1 is a schematic diagram of an active ingredient release system comprising a matrix composed of a biodegradable hydrogel with intercalated active ingredient molecules that are released by degradation of the hydrogel;

FIG. 2 is a schematic diagram of the active ingredient release system comprising a matrix composed of a non-degradable hydrogel with intercalated active ingredient molecules that are released from the hydrogel by diffusion;

FIG. 3 is a schematic of the progression of a coupling reaction of the biodegradable hydrogel onto an optical component of an ophthalmological implant and loading of the hydrogel with an active pharmacological ingredient;

FIG. 4 is a schematic of the progression of a coupling reaction of the non-degradable hydrogel onto an optical component of an ophthalmological implant, where the hydrogel is coupled to the active pharmacological ingredient via degradable bonds;

FIG. 5 is a schematic of the progression of release of the active ingredient present in the degradable hydrogel by biodegradation of the hydrogel; and,

FIG. 6 is a schematic of the progression of release of the active ingredient present in the non-degradable hydrogel.

### DESCRIPTION OF THE PREFERRED EMBODIMENTS

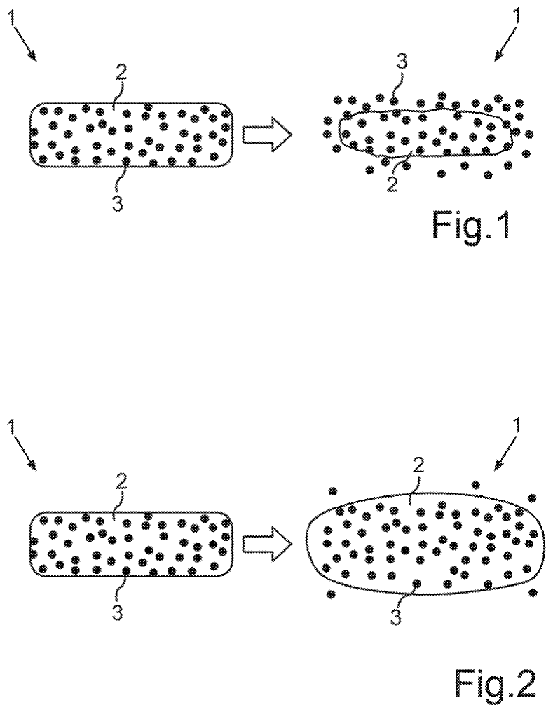

FIG. 1 shows a schematic diagram of an active ingredient release system 1 comprising a matrix composed of a biodegradable hydrogel 2 with intercalated active ingredient molecules 3 that are released into the environment by degradation of the hydrogel 2. The hydrogel 2 is selected, for example, from the group of poly(N-isopropylacrylamide), poly(vinyl alcohol), poly(ethylene glycol), hyaluronic acid, cellulose, poly(lactic acid) and the like. Such hydrophilic long-chain polymers are converted to hydrogels 2, for example, by simple crosslinking reactions. Owing to the large amount of hydrophilic groups, the hydrogel 2 can subsequently absorb a large amount of water, often several times its own starting weight or dry weight. The same interactions are utilized in order to physically “trap” active ingredient molecules 3 within the hydrogel system 2. In the present case, the hydrogel 2 is biodegraded, for example by enzymes that occur in the eye. This releases the previously “trapped” active ingredient molecules 3.

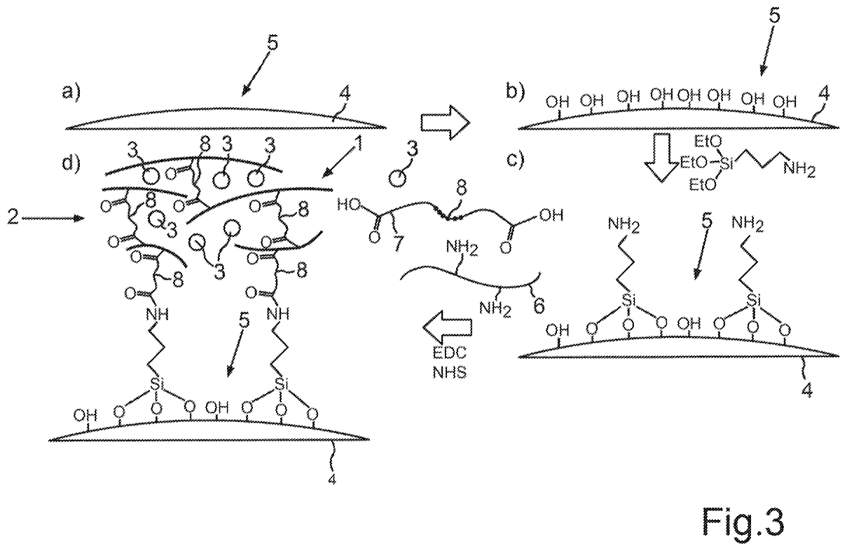

FIG. 2 shows a schematic diagram of the active ingredient release system 1 comprising a matrix composed of a non-degradable hydrogel 2 with intercalated active ingredient molecules 3 that, by contrast with FIG. 1, are released from the hydrogel 2 by diffusion. It can be seen that the hydrogel 2 swells as a result of absorption of additional water, which increases the pore size of the polymer network and facilitates or actually enables the diffusion of the active ingredient molecules 3. For long-lasting controlled release of the active ingredient molecules 3, it is possible to use hydrophilic active ingredient molecules 3 of maximum volume, since they only diffuse slowly out of the hydrogel 2 and hence reduce the likelihood of a burst release a short time after the implantation of an assigned ophthalmological implant 4 (see FIG. 3) into an eye. Small hydrophobic active ingredient molecules 3 are alternatively successfully incorporated into such polymer networks, for example by copolymerization with other monomers and oligomers, or by manipulation of the surface of the hydrogel 2. Alternatively, the active ingredient molecules 3 are also covalently bonded to the hydrogel 2 by bonds that are biodegradable or degradable in some other way. The speed of degradation of these degradable bonds are then used in some embodiments to essentially control the release of the active ingredient molecules 3. All the materials and techniques mentioned for production of medicament-laden hydrogels 2 are used in certain embodiments to appropriately modify the surfaces of optical components 5 and optionally also of tactile components (not shown) of ophthalmological implants 4.

In order to achieve covalent crosslinking of a hydrogel 2 with the surface of the implant 4 via a chemical reaction, it is possible to use various techniques. In an embodiment, reactive groups are grafted by, for example epoxides or thiols, onto the surface of the implant 4. During polymerization of the hydrogel 2 or of the hydrogel precursor 6, parts of the polymer network react with the modified surface, which achieves the immobilization of the hydrogel 2.

This is effected, in an example, via the use of what is called click chemistry on a chemically modified surface of the implant 4. In this way, the target surface of the implant 4 that is to be provided with the hydrogel layer is exposed to a plasma, which forms hydroxyl groups. These can readily react, for example, with triethoxysilanes having different reactive groups, which essentially gives rise to a surface covered completely or predominantly with such reactive groups. According to the type of reaction used, these groups then react directly with the polymers during the hydrogel synthesis. If, for example, an EDC/NHS-mediated amide coupling reaction is used for crosslinking of the hydrogel 2, the grafting of either carboxyl groups or amine groups would allow simultaneous formation of the hydrogel 2 and immobilization thereof on the surface of the implant 4. If, alternatively, a Michael-type thiol reaction is used, the grafting of thiols onto the surface would allow immobilization of the hydrogel 2. Graft reactions with triethoxysilanes allow for the introduction of a multitude of reactive groups on the surface of the implant 4 and allow controlled immobilization in relation to the types of reaction used in the production of the hydrogel 2, and hence high flexibility for preparation of immobilized medicament-eluting hydrogels 2. This offers a wide range of options by which intraocular lenses and other ophthalmological implants 4 modified with hydrogels 2 that release an active ingredient 3 can be produced.

FIG. 3 shows a schematic of the progression of a coupling reaction of the biodegradable hydrogel 2 onto an optical component 5 of an ophthalmological implant 4 and loading of the hydrogel 2 with an active pharmacological ingredient 3. In the example shown, the active ingredient molecules 3 are physically incorporated within the hydrogel 2 during formation. This is achieved in that the implant 4 is first provided in step a) and, in step b), the surface of the optical component 5 of the implant 4 in the region to be coated is provided by plasma activation with hydroxyl groups. These hydroxyl groups are subsequently exposed to reactive silanes, with use in the present case of 3-(triethoxysilyl)propan-1-amine by way of example. In this embodiment, amino groups are grafted therewith. It is generally possible also to use other silanes and other functional groups, provided that the types of reaction required are compatible with one another.

In a next step c), a mixture of a polymer skeleton or precursor 6 of the hydrogel 2 having amino groups, a crosslinker 7 having two (or more) carboxyl groups and optionally at least one degradable bond 8, the active ingredient 3 and EDC/NHS is applied to the surface of the optical component 5. This initiates peptide formation between the crosslinker 7, the precursor 6 and the surface-fixed amino groups, which forms the immobilized hydrogel 2 as the active ingredient release system 1 incorporating the active ingredient molecules 3. For the polymer or precursor 6 itself, it is possible to use any suitable polymer skeleton (for example, poly(ethylene glycol), hyaluronic acid, cellulose, alginate, et cetera), provided that they bear the reactive group(s) required or can be correspondingly modified to bear the reactive group(s) required. If, for example, a carbohydrate is used, it is possible to use a similar EDC/NHS-mediated peptide coupling for this purpose. Alternatively, functional groups may be reversed, in that, for example, carboxyl functions are grafted on and a crosslinker 7 having two (or more) amino groups is used.

The optional degradable bond 8 from the crosslinker 7 is degraded by hydrolysis, for example. For this purpose, in an embodiment, active ester bonds are provided. Alternatively, an enzymatic degradation is used, for example by the use of hyaluronic acid in the polymer skeleton of the hydrogel 2, which is degraded by hyaluronidase in the implanted state of the implant 4. Alternatively or additionally, it is possible to provide peptide bonds that are cleaved by peptidases.

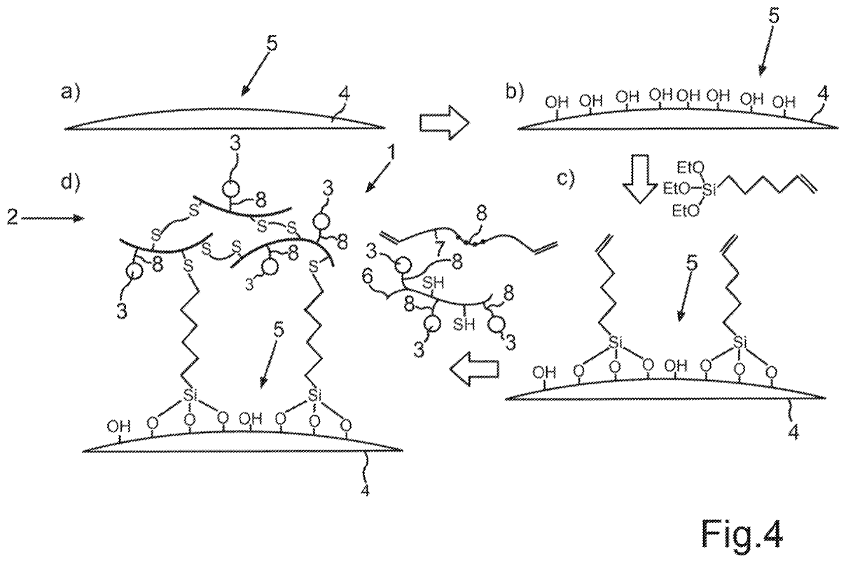

FIG. 4 shows a schematic of the progression of a coupling reaction of the non-degradable hydrogel 2 onto the optical component 5 of the ophthalmological implant 4, where the hydrogel 2 is coupled to the active pharmacological ingredient 3 via degradable bonds 8. In other words, what is used in this embodiment is a non-(bio)degradable hydrogel 2. After plasma activation in step b), this is accomplished by a first grafting of alkene groups onto the surface of the optical component 5 in step c) by the silanization that has already been described. The silane used by way of example is triethoxy(hex-5-enyl)silane. In a next step d), a mixture of thiol-containing polymers or precursors 6 and crosslinkers 7 having two (or more) alkene groups is applied to the modified surface. This induces a Michael-type thiol reaction between the alkenes and thiol groups that leads to the active ingredient release system 1 having the crosslinked surface-immobilized hydrogel 2 and the intercalated active ingredients 3. While the active ingredients 3 in the example shown are physically incorporated within the hydrogel 2 and are subsequently released by diffusion over time, in an alternative embodiment provided are biodegradable bonds 8 in order to covalently bond the active ingredient molecules 3 to the polymer skeleton of the hydrogel 2. This allows for better control over the release profile of the active ingredient 3 via the rate of degradation of these bonds 8. In this way, it is also possible to release relatively small active ingredient molecules 3 over a prolonged period without initial burst release. The (biological/hydrolytic) degradation of the bond 8 between the hydrogel 2 and the active ingredient 3 is significant in determining the release profile.

Similarly to the embodiment described above, any suitable polymer or any suitable precursor 6 is substitutable that is biocompatible and modifiable with the desired chemical groups and is capable of forming hydrogels 2. It is especially possible to use alternative configurations of click chemistry to obtain an immobilized hydrogel 2 with intercalated active ingredient 3. Both embodiments are merely examples of ways in which medicament-eluting hydrogels 2 can be produced as active ingredient release system 1 on surfaces of ophthalmological implants 4, and are not limited to the types of reaction shown in these examples.

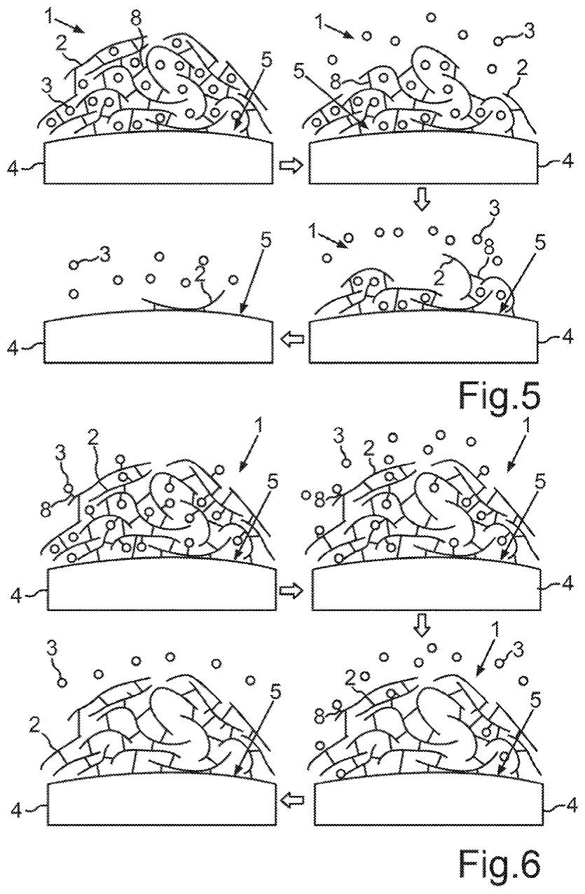

FIG. 5 shows a schematic of the progression of release of the active ingredient 3 present in the degradable hydrogel 2 by biodegradation of the degradable bonds 8 of the hydrogel 2. The rate of degradation of the hydrogel 2 defines the rate of active ingredient release of the active ingredient release system 1.

FIG. 6 shows a schematic of the progression of release of the active ingredient 3 present in the non-degradable hydrogel 2. The active ingredient molecules 3 are bonded to the nondegradable polymer skeleton of the hydrogel 2 via degradable bonds 8. After the degradation of the bonds 8, the active ingredients 3 are released in a diffusion-based manner, which enables particularly long-lasting, uniform release.

What is thus permitted by the use of the active ingredient release systems 1 described for coating of a portion or the entire surface area of the optical component 5 of an ophthalmological implant 4, by comparison with an exclusive arrangement on a tactile component (not shown), is the provision of a great amount of active ingredient without limiting the optical and tactile functionality of the implant 4.

A further advantage relates to reduced tackiness of the surface of the implant 4. A problem with some hydrophobic IOL materials is that their surface is comparatively tacky. The effect of this can be that IOL unfolding can be unfavorable after surgical introduction into the eye. In the extreme case, one or both tactile surfaces can get stuck to the IOL surface of the optical component 5, which requires additional manipulation of the IOL by the doctor. This tack can be avoided by the surface being coated with multiple heparin and polymin layers. However, there are a number of problems with this approach. The use of the active ingredient release system 1 of the invention for partial or complete coating of the optical component 5 also solves the problem of tack and some of the problems with other established coatings.

A further advantageous aspect of the active ingredient release system 1 is improved lubrication. For surgical insertion, intraocular lenses 4 are folded and injected into the eye with an injector. In order to prevent possible damage to the cornea by reduction of the incision size, it is desirable to keep the injection tips as small as possible. This leads to a certain physical force that has to be expended in order to force the folded IOL 4 through the injector. These forces are generally reduced by glide coating of the inner wall of the injector. The immobilized hydrogel 2 of the active ingredient release system 1 may offer (additional) lubrication and either improve the injection process or completely replace the lubrication at the injector wall that has been needed to date.

The active ingredient release system 1 is also used in some embodiments for PCO prevention. A common post-operative complication for cataract operations is “post-operative capsular opacification” (PCO): Cells grow on the surface 5 of the capsular bag and IOL, and cause occlusion of the capsular bag with time. Although this can be treated by application of laser, it is nevertheless desirable to delay the commencement of PCO as far as possible or prevent it completely. The above-described surface modification of an IOL 4 with an immobilized hydrogel 2 that releases active ingredients 3 can slow or completely prevent PCO development. Furthermore, the surface of the hydrogel 2 can optionally be modified in such a way that PCO prevention is additionally assisted, for example, by the grafting of additional functional groups onto the surface of the hydrogel 2. For example, COX-2-blocking NSAIDs can help in the prevention of PCO. Such NSAIDs are used for the treatment of inflammation and can also prevent or considerably slow the development of PCO by being incorporated into the hydrogel 2.

The active ingredient release system 1 is additionally used in some embodiments as a platform for different medicaments and active ingredients 3. As well as the use of inflammation inhibitors as active ingredient 3, it is entirely possible to use the active ingredient release system 1 for uptake and release of other medicament types. This embodiment includes antibiotics for prevention of bacterial infection or medicaments for reduction of intraocular pressure to counter the commencement of glaucoma inter alia. If the hydrogel 2 offers a sufficiently large reservoir, it is also possible to use two or more active ingredients 3 for simultaneous prolonged release.

With regard to hydrophilic IOLs and other hydrophilic ophthalmological implants 4 that are stored in water until implantation, there are various possible strategies for achieving a high storage stability. For example, an implant 4 that has been coated with a non-(bio)degradable hydrogel 2 can be stored in a saturated active ingredient solution. In this way, the active ingredient concentration remains constant during storage within the active ingredient release system 1.

Alternatively or additionally, degradable bonds 8 are incorporated that are not hydrolysis-sensitive. In one example, active ingredients 3 are bound to the hydrogel 2 via degradable peptide bonds 8. Alternatively or additionally, crosslinkers 7 are incorporated that contain peptide bonds and/or generate peptide bonds. Peptidase enzymes in the environment of the eye, after implantation, can then degrade the hydrogel 2 and/or separate the active ingredients 3 from the polymer skeleton of the hydrogel 2. No enzymes are present during storage, which means that the active ingredient release system 1 is storage-stable.

A further variant involves the production or use of a hydrogel 2 that greatly limits the diffusion of the intercalated active ingredients 3, for example via small pore size and large active ingredient molecules 3. The hydrogel 2 is, in an embodiment, produced from an enzymatically degradable component, for example, hyaluronic acid. The active ingredient release system 1 is stable during storage since it is only in the implanted state in the environment of the eye that it is degraded with hyaluronidase (or a comparable enzyme), and the intercalated active ingredients 3 are released.

The parameter values specified in the documents to define process and measurement conditions for the characterization of specific properties of the subject matter described herein are also considered to be encompassed by the scope of the description in the context of deviations—for example due to measurement errors, system errors, weighing errors, DIN tolerances and the like.

It is understood that the foregoing description is that of the preferred embodiments and that various changes and modifications may be made thereto without departing from the spirit and scope of the invention as defined in the appended claims.

### LIST OF REFERENCE NUMERALS

1 Active ingredient release system
2 Hydrogel
3 Active ingredient
4 Implant
5 Optical component
6 Precursor
7 Crosslinker
8 Degradable bond

What is claimed is:

1. An ophthalmological implant, comprising:
at least one hydrogel as a matrix, and
at least one optical component,
wherein the at least one hydrogel forms a layer on the optical component of the implant, is covalently bonded to the optical component, and is laden with at least one pharmacologically active ingredient, and
wherein the at least one pharmacologically active ingredient is released when the ophthalmological implant is implanted into a tissue of a subject.

2. The ophthalmological implant as claimed in claim 1,
wherein the ophthalmological implant is an intraocular lens.

3. The ophthalmological implant as claimed in claim 1,
wherein the at least one hydrogel is degradable.

4. The ophthalmological implant as claimed in claim 1,
wherein the hydrogel is comprised of any one or more of:
poly(N-isopropylacrylamide), polyvinylalcohol, polyethyleneglycol, polylactic acid, polyethyleneimine, cellulose, cellulose ethers having methyl and/or ethyl and/or propyl groups, especially hydroxypropyl methylcellulose, hydroxyethyl methylcellulose and/or methylcellulose, glycosaminoglycans, especially hyaluronic acid, chondroitin sulfate, dermatan sulfate, heparin, heparan sulfate, keratan sulfate, alginic acid, polymannuronic acid, polyguluronic acid, polyglucuronic acid, amylose, amylopectin, callose, chitosan, polygalactomannan, dextran, xanthan, a mixture, and/or a physiologically acceptable salt thereof.

5. The ophthalmological implant as claimed in claim 1,
wherein the at least one active ingredient is:
covalently bonded to the hydrogel via a biodegradable bond,
covalently bonded to a monomer, and/or oligomer,
distributed in the at least one hydrogel, and/or
resides in polymer nanoparticles distributed within the at least one hydrogel that are biodegradable.

6. The ophthalmological implant as claimed in claim 1,
wherein hydrogel comprises at least one surface region, and wherein the at least one pharmacologically active ingredient is covalently bonded to the at least one surface region of the at least one hydrogel.

7. The ophthalmological implant as claimed in claim 1,
wherein the at least one pharmacologically active ingredient is one or more of a steroidal inflammation inhibitor, a non-steroidal inflammation inhibitor, a prostaglandin, a prostamide, an antibiotic, and a beta-blocker.

8. The ophthalmological implant as claimed in claim 1,
wherein the ophthalmological implant is stored in a saturated solution of the at least one active ingredient.

9. A process for producing an ophthalmological implant, which comprises:
providing an active ingredient release system,
wherein the active ingredient release system releases at least one pharmacologically active ingredient when the ophthalmological implant is implanted into a subject,
providing an optical component,
coating the optical component with at least one hydrogel as matrix of the active ingredient release system, and
covalently binding the at least one hydrogel to the optical component, wherein the at least one hydrogel is laden with the at least one active ingredient.

10. The process as claimed in claim 9,
wherein covalently binding comprises:
generating surface hydroxyl groups on the optical component;
graft polymerizing at least one reactive silane compound comprising at least one functional group and a silane group onto the surface hydroxyl groups; and
covalently binding the at least one hydrogel onto the at least one functional group of the grafted silane compound.

11. The ophthalmological implant as claimed in claim 2, wherein the intraocular lens is an accommodating intraocular lens, ring, or tube.

12. The ophthalmological implant as claimed in claim 11, wherein the intraocular lens is an accommodating ring, and wherein the ring is a capsular tension ring.

13. The ophthalmological implant as claimed in claim 1, wherein the hydrogel is biodegradable.

14. The ophthalmological implant as claimed in claim 1, wherein the at least one pharmacologically active ingredient is a COX-2 inhibitor.
